(19) United States
(12) Patent Application Publication
(10) Pub. No.: US 2026/0306362 A1
(43) Pub. Date: Oct. 8, 2026

(54) Syringe Adapter With Membrane Housing

(71) Applicant: Becton, Dickinson and Company, Franklin Lakes, NJ (US)

(72) Inventors: Rafael Gonzalez Macias, San Diego, CA (US); Nicole Jean DiStefano, Dumont, NJ (US)

(21) Appl. No.: 19/484,985

(22) PCT Filed: May 13, 2024
(86) PCT No.: PCT/US2024/029047

§ 371 Date: Nov. 17, 2025

Related U.S. Application Data
(60) Provisional application No. 63/468,896, filed on May 25, 2023.

(51) Int. Cl. A61J 1/20

(52) U.S. Cl. CPC A61J 1/2051; A61J 1/2065

(57) ABSTRACT

A syringe adapter includes an outer housing having a first end and a second end positioned opposite the first end with a longitudinal axis extending from the first end of the outer housing to the second end of the outer housing, with the outer housing including a sidewall defining an interior space, a syringe connection configured to be connected to a syringe, and a membrane housing positioned within the interior space of the outer housing and moveable from a first position within the outer housing to a second position within the outer housing spaced from the first position. The membrane housing receives a membrane. The membrane housing includes a biasing member configured to engage the sidewall of the outer housing, and the biasing member configured to provide a biasing force in a direction perpendicular to the longitudinal axis of the outer housing.

### CROSS-REFERENCE TO RELATED APPLICATION

The present application claims priority to U.S. Provisional Application No. 63/468,896 entitled “Syringe Adapter with Membrane Housing”, filed May 25, 2023, the disclosure of which is hereby incorporated by reference in its entirety.

### BACKGROUND OF THE INVENTION

### Field of the Invention

The present invention relates to a syringe adapter with a membrane housing.

### Description of Related Art

Health care providers reconstituting, transporting, and administering hazardous drugs, such as chemotherapeutic medication, can put health care providers at risk of exposure to these medications and present a hazard in the health care environment. Unintentional chemotherapy exposure can affect the nervous system, impair the reproductive system, and bring an increased risk of developing blood cancers in the future. Some drugs must be dissolved or diluted before they are administered, which involves transferring a solvent from one container to a sealed vial containing the drug in powder or liquid form. Drugs may be inadvertently released into the atmosphere in gas form or by way of aerosolization, during the withdrawal of the medication from the vial if any pressure differential between the interior of the vial and the surrounding atmosphere exists. In order to reduce the risk of health care providers being exposed to toxic drugs, the transfer of these drugs is accomplished utilizing a closed system transfer device or system.

Syringe adapters are utilized with a syringe to transfer medication from a container, such as a vial, to the syringe or to transfer a diluent from the syringe to the vial to reconstitute a medication. Syringe adapters are also utilized to transfer medication from the syringe to a patient infusion line or to an infusion container, such as an IV bag, via an IV bag spike. Syringe adapters include a membrane received by a membrane housing, with the membrane of the syringe adapter configured to engage a corresponding membrane of a mating component, such as a patient connector, vial adapter, or IV bag spike, to ensure the closed transfer of fluid between the components such that the medication is not exposed to the ambient environment.

### SUMMARY OF THE INVENTION

In one aspect or embodiment, a syringe adapter includes an outer housing having a first end and a second end positioned opposite the first end with a longitudinal axis extending from the first end of the outer housing to the second end of the outer housing, with the outer housing including a sidewall defining an interior space, a syringe connection configured to be connected to a syringe, and a membrane housing positioned within the interior space of the outer housing and moveable from a first position within the outer housing to a second position within the outer housing spaced from the first position. The membrane housing receives a membrane. The membrane housing includes a biasing member configured to engage the sidewall of the outer housing, the biasing member configured to provide a biasing force in a direction perpendicular to the longitudinal axis of the outer housing.

An inner diameter of the outer housing may be larger at the second end of the outer housing than the inner diameter of the outer housing at the first end of the housing, where the biasing member engages the sidewall of the outer housing as the membrane housing moves from the first position to the second position. The biasing member of the membrane housing may be configured to bias the membrane housing toward the first position as the membrane housing moves from the first position to the second position. The inner diameter of the outer housing may taper from the first end of the outer housing to the second end of the outer housing. The biasing member may extend radially outward from the membrane housing. The biasing member may include a plurality of arcuate projections. Each of the plurality of arcuate projections may include a first end connected to the membrane housing, a second end positioned opposite to the first end, and a contact surface configured to engage an inner surface of the sidewall of the outer housing. The contact surface of each of the plurality of arcuate projections may have a radius equal to a radius of a portion of the inner surface of the sidewall of the outer housing. The biasing member may be configured to center the membrane housing within the interior space of the outer housing. The membrane housing may have a first end and a second end positioned opposite the first end of the membrane housing, where the biasing member is positioned between the first and second ends of the membrane housing. The biasing member may be positioned closer to the first end of the membrane housing than the second end of the membrane housing.

The syringe adapter may further include a needle received within the interior space of the outer housing. A piercing tip of the needle may be positioned within the membrane when the membrane housing is in the first position, where the piercing tip of the needle is positioned outside of the membrane when the membrane housing is in the second position. The membrane housing may include a collet configured to receive a connection interface of a mating connector.

### BRIEF DESCRIPTION OF THE DRAWINGS

FIG. 1 is a front view of a conventional syringe adapter.

FIG. 2 is a cross-sectional view of the syringe adapter of FIG. 1.

FIG. 3 is a perspective view of a membrane housing according to one aspect or embodiment of the present application.

FIG. 4 is a top view of the membrane housing of FIG. 3.

FIG. 5 is a cross-sectional view of the membrane housing of FIG. 3, showing the membrane housing engaged with a syringe adapter housing.

### DETAILED DESCRIPTION

For purposes of the description hereinafter, the terms such as “end”, “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting. Further, it is to be understood that the invention may assume various alternative variations and step sequences, except where expressly specified to the contrary.

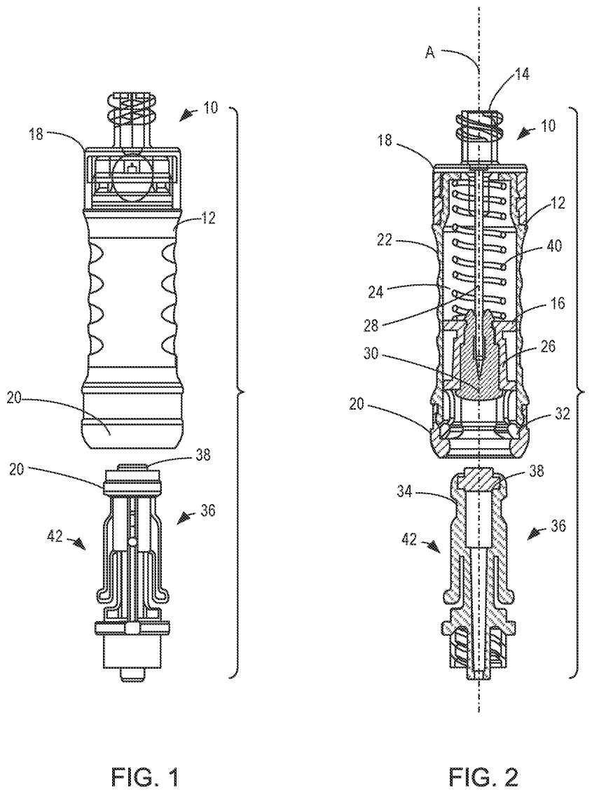

Referring to FIGS. 1 and 2, a conventional syringe adapter 10 is shown. The syringe adapter 10 is one component of a system for the closed transfer of fluids. In particular, the syringe adapter 10 is configured to connect to a syringe (not shown) and to another medical device or fluid container. The medical device can be, for example, a patient line, vial adapter, fluid container, or infusion adapter. The container can be a medical vial, syringe barrel, IV bag, or similar container for holding a fluid to be administered to a patient. The syringe adapter 10 can be used to facilitate the closed transfer of fluids between the syringe and medical device or fluid container. The syringe adapter 10 is shown and described in U.S. Patent Application Publication No. 2018/0200147, which is hereby incorporated by reference in its entirety.

Referring again to FIGS. 1 and 2, the syringe adapter 10 includes an outer housing 12, a syringe connection 14, and a membrane housing 16. The outer housing 12 has a first end 18 and a second end 20 positioned opposite the first end 18 with a longitudinal axis A extending from the first end 18 of the outer housing 12 to the second end 20 of the outer housing 12. The outer housing 12 includes a sidewall 22 defining an interior space 24. The syringe connection 14 is configured to be connected to a syringe. The syringe connection 14 may be a luer connector, although other suitable connection arrangements may be utilized. The membrane housing 16 is positioned within the interior space 24 of the outer housing 12 and is moveable from a first position within the outer housing 12 to a second position within the outer housing 12 that is spaced from the first position. The membrane housing 12 receives a membrane 26. The syringe adapter 10 further includes a needle 28 received within the interior space 24 of the outer housing 12. A piercing tip 30 of the needle 28 is positioned within the membrane 26 when the membrane housing 16 is in the first position and the piercing tip 30 of the needle 28 is positioned outside of the membrane 26 when the membrane housing 16 is in the second position. The membrane housing 16 includes a collet 32 configured to receive a connection interface 34 of a mating connector 36, such as a patient connecter as shown in FIGS. 1 and 2. The mating connector 36 includes a membrane 38. The mating connector 36 may be other components of a closed system transfer device or system, such as a vial adapter or IV bag spike. The syringe adapter 10 also includes a spring 40 received within the interior space 24 of the outer housing 12. The spring 40 biases the membrane housing 16 toward the first position.

When the mating connector 36 is inserted into the outer housing 16 of the syringe adapter 10, the collet 32 of the membrane housing 16 receives the connection interface 34 of the mating connector 36 and moves the membrane housing 16 within the outer housing 12 toward the first end of the outer housing 12. As the membrane housing 16 moves, the collet 32 secures the membrane housing 16 to the mating connector 36 to ensure engagement between the membrane 38 of the mating connector 36 and the membrane 26 of the membrane housing 16. As the membrane housing 16 is moved from the first position to the second position, the piercing tip 30 of the needle 28 pierces the membrane 26 of the membrane housing 16 and the membrane 38 of the mating connector 36 such that the piercing tip 30 is received within the mating connector 36 thereby placing the needle 28 in fluid communication with the mating connector 36. The syringe connection 14 is in fluid communication with the needle 28 to allow a syringe (not shown) connected to the syringe connection 14 to be in fluid communication with the mating connector 36 when the syringe adapter 10 is connected to the mating connector 36. Moving the membrane housing 16 from the second position to the first position, which is aided by the spring 40, releases the mating connector 36 from the collet 32 to separate the mating connector 36 from the syringe adapter 10. The membrane housing 16 is secured in the second position by a locking arrangement 42 of the mating connector 36 that engages a portion of the outer housing 12 to hold the membrane housing 16 in the second position. Pulling the mating connector 36 away from the syringe adapter 10 releases the locking arrangement 42 to allow the membrane housing 16 to move back to the first position from the second position.

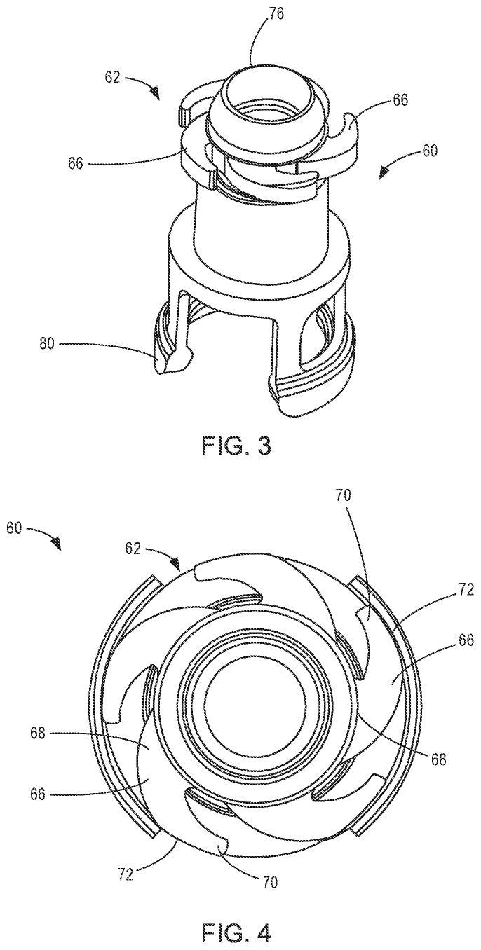

Referring to FIGS. 3-5, in accordance with one aspect or embodiment, the syringe adapter 10 includes a membrane housing 60 having a biasing member 62 configured to engage the sidewall 22 of the outer housing 12. The biasing member 62 is configured to provide a biasing force in a direction perpendicular to the longitudinal axis A of the outer housing 12. The membrane housing 60 receives the membrane 26 and may be utilized with the syringe adapter 10 shown in FIGS. 1 and 2 and operate in the same manner as the membrane housing 16 discussed above, except for the differences discussed below. The membrane housing 60 of FIGS. 3-5 may also be utilized in connection with other types of syringe adapters, such as syringe adapters not including the spring or not including the needle. The biasing member 62 of the membrane housing 60 is configured to automatically center the membrane housing 60 within the interior space 24 of the outer housing 12 to ensure consistent operation of the syringe adapter 10. The biasing member 62 of the membrane housing 60 is configured to compensate for dimension variations in the parts of the syringe adapter 10 to ensure consistent functionality.

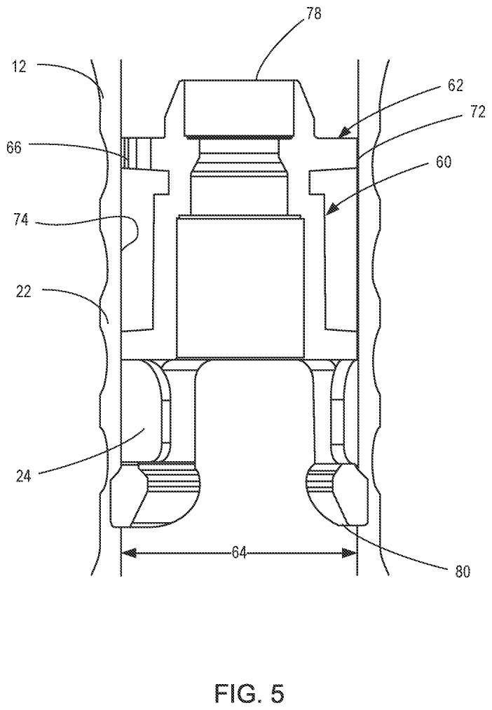

Referring to FIG. 5, in one aspect or embodiment, an inner diameter 64 of the outer housing 12 is larger at the second end 20 of the outer housing 12 than an inner diameter of the outer housing 12 at the first end 18 of the outer housing 12. The biasing member 62 engages the sidewall 22 of the outer housing 12 as the membrane housing 60 moves from the first position to the second position. The biasing member 62 of the membrane housing 60 is configured to bias the membrane housing 60 toward the first position as the membrane housing 60 moves from the first position to the second position. In one aspect or embodiment, the biasing member 62 eliminates the need for the spring 40 and returns the membrane housing 60 to the first position from the second position. The inner diameter 64 of the outer housing 12 tapers from the first end 18 of the outer housing 12 to the second end 20 of the outer housing 12, although other suitable arrangements may be utilized. The biasing force provided by the biasing member 62 may increase as the membrane housing 60 moves toward the second position closer to the first end 18 of the outer housing 12. The biasing member 62 extends radially outward from the membrane housing 60. In one aspect or embodiment, the biasing member 62 is formed integrally with the membrane housing 60, although the biasing member 62 may be formed separately from the membrane housing 60 and may be secured to the membrane housing 60 in any suitable manner. As shown in FIGS. 3 and 4, the biasing member 62 is a plurality of arcuate projections 66, although one or more arcuate projections 66 may be utilized. As shown in FIG. 4, the biasing member 62 includes five equally-spaced arcuate projections 66.

Each of the plurality of arcuate projections 66 includes a first end 68 connected to the membrane housing 60, a second end 70 positioned opposite to the first end 68, and a contact surface 72 configured to engage an inner surface 74 of the sidewall 22 of the outer housing 12. The contact surface 72 of each of the plurality of arcuate projections 66 has a radius equal to a radius of a portion of the inner surface 74 of the sidewall 22 of the outer housing 12. The contact surface 72 of each of the plurality of arcuate projections 66 is curved, although other suitable arrangements may be utilized. The membrane housing 60 has a first end 78 and a second end 80 positioned opposite the first end 78 of the membrane housing 60. The biasing member 62 is positioned between the first and second ends 78, 80 of the membrane housing 60. The biasing member 62 is positioned closer to the first end 78 of the membrane housing 60 than the second end 80 of the membrane housing 60, although other suitable positions may be utilized.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. To the extent possible, one or more features of any aspect or embodiment discussed above can be combined with one or more features of any other aspect or embodiment.

1. A syringe adapter comprising:
an outer housing having a first end and a second end positioned opposite the first end with a longitudinal axis extending from the first end of the outer housing to the second end of the outer housing, the outer housing includes a sidewall defining an interior space;
a syringe connection configured to be connected to a syringe; and
a membrane housing positioned within the interior space of the outer housing and moveable from a first position within the outer housing to a second position within the outer housing spaced from the first position, the membrane housing receiving a membrane, wherein the membrane housing comprises a biasing member configured to engage the sidewall of the outer housing, the biasing member configured to provide a biasing force in a direction perpendicular to the longitudinal axis of the outer housing.

2. The syringe adapter of claim 1, wherein an inner diameter of the outer housing is larger at the second end of the outer housing than the inner diameter of the outer housing at the first end of the housing, and wherein the biasing member engages the sidewall of the outer housing as the membrane housing moves from the first position to the second position.

3. The syringe adapter of claim 2, wherein the biasing member of the membrane housing is configured to bias the membrane housing toward the first position as the membrane housing moves from the first position to the second position.

4. The syringe adapter of claim 2, wherein the inner diameter of the outer housing tapers from the first end of the outer housing to the second end of the outer housing.

5. The syringe adapter of claim 1, wherein the biasing member extends radially outward from the membrane housing.

6. The syringe adapter of claim 5, wherein the biasing member comprises a plurality of arcuate projections.

7. The syringe adapter of claim 6, wherein each of the plurality of arcuate projections comprises a first end connected to the membrane housing, a second end positioned opposite to the first end, and a contact surface configured to engage an inner surface of the sidewall of the outer housing.

8. The syringe adapter of claim 7, wherein the contact surface of each of the plurality of arcuate projections has a radius equal to a radius of a portion of the inner surface of the sidewall of the outer housing.

9. The syringe adapter of claim 1, wherein the biasing member is configured to center the membrane housing within the interior space of the outer housing.

10. The syringe adapter of claim 1, wherein the membrane housing has a first end and a second end positioned opposite the first end of the membrane housing, wherein the biasing member is positioned between the first and second ends of the membrane housing.

11. The syringe adapter of claim 10, wherein the biasing member is positioned closer to the first end of the membrane housing than the second end of the membrane housing.

12. The syringe adapter of claim 1, further comprising a needle received within the interior space of the outer housing.

13. The syringe adapter of claim 12, wherein a piercing tip of the needle is positioned within the membrane when the membrane housing is in the first position, and wherein the piercing tip of the needle is positioned outside of the membrane when the membrane housing is in the second position.

14. The syringe adapter of claim 1, wherein the membrane housing comprises a collet configured to receive a connection interface of a mating connector.
